(19) United States
(12) Patent Application Publication
(10) Pub. No.: US 2004/0132079 A1
(43) Pub. Date: Jul. 8, 2004

(54) Assays relating to Toll-like receptor activity

(75) Inventors: Shalley K. Gupta, Woodbury, MN (US); Tarun K. Ghosh, Woodbury, MN (US); Jason R. Fink, Eagan, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/732,563

(22) Filed: Dec. 10, 2003

Related U.S. Application Data
(60) Provisional application No. 60/432,650, filed on Dec. 11, 2002.

(51) Int. Cl. C12Q 1/68; G01N 33/53; G01N 33/567; C12N 15/85

(52) U.S. Cl. 435/6; 435/7.2; 435/455

(57) ABSTRACT

The present invention provides assays useful for detecting agonists of Toll-like receptors. The assays include, generally, providing a cell culture transfected with a nucleic acid sequence that encodes a reporter operably linked to a TLR-inducible expression control sequence; contacting the cell culture with a test compound; and identifying the compound as a TLR agonist if TLR-inducible expression of the reporter can be detected.

### CROSS-REFERENCE TO RELATED APPLICATION

This application claims priority to U.S. Provisional Patent Application No. 60/432,650, filed Dec. 11, 2002.

### BACKGROUND OF THE INVENTION

Cells of the immune system secrete a diverse set of compounds including cytokines, chemokines, co-stimulatory markers, and defensins in response to an immunological challenge.

Certain compounds known as immune response modifiers (“IRMs”) possess potent immunostimulating activity including but not limited to antiviral and antitumor activity. Certain IRMs act by, e.g., inducing the production and secretion of certain cytokines while inhibiting production and secretion of other cytokines.

Certain IRMs are small organic molecules (e.g., molecular weight under about 1000 Daltons, preferably under about 500 Daltons, as opposed to large biologic protein, peptides, and the like) such as those disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,545,016; 6,545,017; 6,558,951; and 6,573,273; European Patent 0 394 026; U.S. Patent Publication No. 2002/0055517; and International Patent Publication Nos. WO 01/74343; WO O2/46188; WO O2/46189; WO O2/46190; WO O2/46191; WO O2/46192; WO O2/46193; WO O2/46749 WO O2/102377; WO 03/020889; WO 03/043572 and WO 03/045391.

Additional examples of small molecule IRMs include certain purine derivatives (such as those described in U.S. Pat. No. 6,376,501, and 6,028,076), certain imidazoquinoline amide derivatives (such as those described in U.S. Pat. No. 6,069,149), certain benzimidazole derivatives (such as those described in U.S. Pat. No. 6,387,938), and certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO O2/085905).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG and are described, for example, in International Patent Publication No. WO 00/75304.

Some of these IRMs induce cellular responses (e.g., the production and/or secretion of cytokines, chemokines, etc.) through one or more Toll-like receptors (TLRs). For example, certain small organic molecule IRMs are agonists of one or more of TLR1, TLR2, TLR4, TLR6, TLR7, and TLR8. Additionally, CpG has been reported to act through TLR9.

In certain cells of the immune system, TLR activation can be associated with activation of the transcription factor NF-κB. NF-κB activation is associated with certain cellular responses to an immunological challenge, such as the production and secretion of pro-inflammatory cytokines such as TNF-α, IL-1, IL-6, IL-8, IL-10, IL-12, MIP-1, and MCP-1. IRM induction of such cellular responses can be demonstrated by measuring activation of the transcription factor NF-κB in response to exposing a cell to an IRM compound (See, e.g., Chuang et al., Journ. of Leuk. Biol., vol. 71, pp. 538-544 (2002), and Hemmi et al., Nature Immunology, vol. 3(2), pp. 196-200 (2002)). Thus, NF-κB activation can be used as a reporter of TLR activation. However, the extent of NF-κB activation does not necessarily correlate with the extent of the downstream cellular response. This is so because the downstream cellular response may be modulated by one or more additional factors.

### SUMMARY OF THE INVENTION

The present invention provides assays for detecting activation of a TLR. The assays include providing a cell culture comprising cells transfected with a nucleic acid sequence that encodes a reporter that (a) generates a detectable signal when the reporter is expressed and the cell is exposed to conditions effective for generating the detectable signal, and (b) is operably linked to an expression control sequence that is induced by activation of a TLR and comprises a cytokine promoter, a chemokine promoter, a co-stimulatory marker promoter, or a defensin promoter; exposing the cell culture to a compound that activates a TLR; providing conditions effective for generating the detectable signal; and detecting the detectable signal.

In another aspect, the present invention provides assays for identifying agonists of a TLR. The assays include providing a cell culture comprising cells transfected with a first nucleic acid sequence that comprises a nucleotide sequence that encodes a TLR operably linked to a first expression control sequence, and a second nucleic acid sequence that encodes a reporter that (a) generates a detectable signal when the reporter is expressed and the transfected cell is exposed to conditions effective for generating the detectable signal, and (b) is operably linked to a second expression control sequence that is induced by activation of a TLR; contacting the cell culture with a test compound; providing conditions effective for generating the detectable signal, thereby generating a TLR-mediated detectable signal; and identifying the compound as an agonist of the TLR if a TLR-mediated detectable signal is detected.

In another aspect, the present invention provides assays for identifying antagonists of a TLR. These assays include providing a cell culture that comprises cells transfected with a first nucleic acid sequence that comprises a nucleotide sequence that encodes the TLR operably linked to a first expression control sequence, and a second nucleic acid sequence that encodes a reporter that (a) is operably linked to a second expression control sequence that is induced by activation of a TLR, and (b) generates a detectable signal when the reporter is expressed and the transfected cell is exposed to conditions effective for generating the detectable signal; contacting the cell culture with an agonist of the TLR and a test compound; providing conditions effective for generating the detectable signal, thereby permitting the cell culture to generate a full TLR-mediated detectable signal in the absence of an antagonist of the TLR; measuring the detectable signal; and identifying the compound as an antagonist of the TLR if the detectable signal is less than a full TLR-mediated detectable signal.

In another aspect, the present invention provides a TLR agonists and TLR antagonists identified using an assay according to certain embodiments of the present invention.

In yet another aspect, the present invention provides pharmaceutical compositions including a TLR agonist or a TLR antagonist identified using an assay according to certain embodiments of the present invention.

Various other features and advantages of the present invention should become readily apparent with reference to the following detailed description, examples, and claims. In several places throughout the specification, guidance is provided through lists of examples. In each instance, the recited list serves only as a representative group and should not be interpreted as an exclusive list.

### DETAILED DESCRIPTION OF ILLUSTRATIVE EMBODIMENTS OF THE INVENTION

The present invention provides assays that may be useful for detecting TLR activation based on detecting induction of a downstream cellular response to TLR activation (e.g., production or secretion of one or more immune system compounds such as cytokines or co-stimulatory markers) rather than NF-κB activation. In some cases, the cellular response may be mediated by NF-κB, but in other cases the cellular response may be NF-κB-independent. Thus, the present invention provides assays that may be useful for detecting a broader range of TLR activation than is possible by monitoring NF-κB activation. This may provide an ability to identify certain TLR agonists that would not be detected using an assay based on NF-κB activation. The assays of the present invention also may provide a more relevant indication of the quantitative character of a particular cellular response to TLR activation by a particular TLR agonist.

In some cases, an assay according to the present invention may be useful for detecting TLR activation that is not accompanied by NF-κB activation. Such an assay may be employed to identify TLR agonists that do not necessarily also activate NF-κB. Such TLR agonists may be useful for treatment or prevention of certain conditions in which the production and secretion of pro-inflammatory cytokines such as those induced by NF-κB activation may be undesirable.

For purposes of this invention, the following terms shall have the meanings set forth.

“Activation” refers to modifying the indicated protein so that the protein provides a biological function. For example, TLR activation refers to modifying a TLR—for example, a conformational modification such as in response to exposure of the TLR to an agonist—so that the TLR is capable of inducing the production and secretion of certain cytokines.

“Agonist” refers to a compound that can combine with a receptor (e.g., a TLR) to produce a cellular response. An agonist may be a ligand that directly binds to the receptor. Alternatively, an agonist may combine with a receptor indirectly by, e.g., (a) forming a complex with another molecule that directly binds to the receptor, or (b) otherwise results in the modification of another compound so that the other compound directly binds to the receptor. An agonist may be referred to as an agonist of a particular TLR (e.g., a TLR6 agonist).

“Amino acid sequence” refers to a particular ordered sequence of amino acids, whether naturally occurring or engineered.

“Antagonist” refers to a compound that can combine with a receptor (e.g., a TLR) to inhibit a cellular response. An antagonist may be a ligand that directly binds to the receptor. Alternatively, an antagonist may combine with a receptor indirectly by, e.g., (a) forming a complex with another molecule that directly binds to the receptor, or (b) otherwise results in the modification of another compound so that the other compound directly binds to the receptor. An antagonist may be referred to as an antagonist of a particular TLR (e.g., a TLR6 antagonist). An antagonist may inhibit biological activity to any measurable extent.

“Co-transfect” and variations thereof refer to transfecting a host cell with more than one vector. A host cell may be co-transfected by transfecting with two or more vectors one at a time or in any convenient combination of vectors, including simultaneous transfection with all vectors.

“Express” and variations thereof refer to the ability of a cell to transcribe a structural gene to mRNA, then translate the mRNA to synthesize a protein that provides a detectable biological or biochemical function. “Expressible” refers to the ability of a particular nucleic acid sequence to be expressed by a cell that contains the nucleic acid sequence.

“Immune system compound” refers to any compound that is produced or secreted by cells of the immune system in response to an immunological challenge. Immune system compounds include but are not limited to cytokines, chemokines, co-stimulatory markers, and defensins.

“Inhibit” refers to any measurable reduction of biological activity.

“IRM compound” refers to a compound that alters the level of one or more immune system compounds when administered to an IRM-responsive cell. Representative IRM compounds include the small organic molecules, purine derivatives, small heterocyclic compounds, amide derivatives, and oligonucleotide sequences described above.

“Nucleic acid sequence” refers generally to a region of DNA that has a definable function such as (a) encoding a peptide, polypeptide, or protein or (b) controlling expression of a nucleic acid sequence that encodes a peptide, polypeptide, or protein. For example, a nucleic acid sequence that encodes TLR6 refers generically to any sequence of nucleotides that encodes a TLR6 protein, without regard to (a) the species source of the nucleic acid sequence, (b) specific nucleotide sequence variants, or (c) whether such nucleotide sequence variants are naturally occurring or engineered.

“Nucleotide sequence” refers to a particular ordered sequence of nucleotide bases, whether naturally occurring or engineered. “TLR-mediated detectable signal” refers to a detectable signal or that portion of a detectable signal that is attributable to activation of a TLR expressed from a gene expression system transfected into a host cell. For example, a host cell may naturally generate a background level detectable signal (S₀), but generate a greater detectable signal (S_{T}) after being transfected with, and then expressing, a nucleic acid sequence that encodes a TLR. Thus, the TLR-mediated detectable signal (S_{TLR}) refers to the portion of the detectable signal generated by the transfected cell that is greater than background: S_{TLR}=S_{T}−S₀.

It has been found that induction of certain secreted proteins or polypeptides can be useful as reporters of TLR activation. For example, IFN-α is a cytokine secreted by such immune system cells as T lymphocytes, macrophages, plasmacytoid monocytes, dendritic cells, and natural killer cells. IFN-α is involved in regulating a host's innate and adaptive immune responses to an immunological challenge, perhaps by providing a link between the two responses [Brassard et al., Journal of Leukocyte Biology 71: 565-581 (2002)]. The innate immune response can include the cell-mediated response of natural killer (NK) cells to a non-self (e.g., neoplastic) or foreign (e.g., viral) antigen. IFN-α also may indirectly regulate the balance between Th 1 and Th2 cell populations and, therefore, the innate and adaptive immune responses. Moreover, induction of IFN-α is independent of NF-κB activation.

Additionally, the production and secretion of NF-κB-dependent cytokines can be useful as reporters of cellular responses resulting from immunological challenge. Detection and measurement of such cytokines may provide comparative qualitative data regarding a cell's response to immunological challenge that is more relevant to an investigator than NF-κB activation data.

Thus, the present invention relates to assays designed to detect induction of immune system compounds. Such assays also may be useful for identifying compounds that induce expression of immune system compounds through TLRs.

Parts of the following description are provided in the context of IFN-α induction and detection. However, many of the features of the embodiments described below also may be realized using assays designed to specifically detect or induce other immune system compounds. Thus, assays designed to specifically detect or induce immune system compounds other than IFN-α are explicitly included in the scope of the present invention.

Assay Tools

The assays of the present invention employ a recombinant cell line capable of inducing gene expression from an expression control sequence of a gene that encodes an immune system compound (e.g., IFN-α) in response to TLR activation. In some embodiments, for example, cells of the recombinant cell line, when exposed to a TLR agonist, can induce expression from an IFN-α promoter to a greater extent than cells of the corresponding untransfected cell line. Cells of the untransfected cell lines may substantially lack a functional level of TLR expression (i.e., untransfected cells may not detectably induce expression from the IFN-α promoter in response to exposure to a TLR agonist). Alternatively, cells of the untransfected cell line may exhibit a baseline level of background TLR function, but the baseline level is less than the level of TLR function observed in cells of the corresponding recombinant (i.e., transfected) cell line.

Cells of certain recombinant cell lines include a first nucleic acid sequence that encodes a TLR operably linked to an expression control sequence. The cells also include a second nucleic acid sequence that encodes a reporter capable of generating a detectable signal when it is expressed in the recombinant cell under conditions suitable for generating the detectable signal. The reporter is linked to a second expression control sequence that is capable of being induced by activation of the TLR encoded by the first nucleic acid sequence.

The TLR encoded by the first nucleic acid sequence, when present, may be any TLR. Ten different human TLRs have been identified, cloned, and sequenced. TLRs also are known to exist in other mammals including, for example, mice and chimpanzees. The nucleotide sequences of the ten human TLRs and many non-human TLRs are known, have been published, and are readily accessible from various sequence databases including GenBank. The first nucleic acid sequence may include the nucleotide sequence for any one of the TLRs, whether human or non-human. In one embodiment, the TLR is human TLR6; in another embodiment, the TLR is human TLR7. Alternatively, the first nucleic acid may encode any one of the ten human TLRs, any non-human TLR, or any combination of two or more TLRs that may be desirable for a particular construct.

The first nucleic acid sequence, when present, can include a nucleotide sequence that differs from a specific published nucleotide sequence for the TLR encoded by the first nucleic acid sequence. For example, the first nucleic acid sequence can contain one or more substitutions (compared to a published TLR nucleotide sequence) that do not alter the amino acid sequence of the TLR protein expressed from the first nucleic acid sequence. Such a substitution may be termed a degenerate substitution. Nucleotide sequences containing one or more degenerate substitutions compared to a known TLR nucleotide sequence are explicitly included within the scope of nucleotide sequences suitable for use within the first nucleic acid sequence.

As another example, certain nucleotide substitutions may alter the amino acid sequence of the TLR protein. For certain amino acid substitutions, however, the chemical properties of the protein having the altered amino acid sequence are similar to the chemical properties of the protein having the native amino acid sequence. Amino acids may be divided into four groups based on the chemical characteristics of the amino acid side groups: neutral, non-polar amino acids include glycine, alanine, valine, isoleucine, leucine, phenylalanine, proline, and methionine; neutral, polar amino acids include serine, threonine, tyrosine, tryptophan, asparagine, glutamine, and cysteine; acidic amino acids include aspartic acid and glutamic acid; and basic amino acids include lysine, arginine, and histidine. Substitution of one amino acid for another amino acid within the same group may have little or no functional effect on the resulting protein because of the similarity of the chemical characteristics of the amino acids involved in the substitution. Such amino acid substitutions may be termed a conservative amino acid substitution. Nucleotide sequences that, when compared to a known TLR nucleotide sequence, generate one or more conservative amino acid substitutions are explicitly included within the scope of nucleotide sequences suitable for use within the first nucleic acid sequence.

The nucleic acid sequence that encodes a TLR, if present, may be cloned into an expression vector so that it is under the expression control of its own promoter, a homologous TLR promoter, or any heterologous promoter inducible in an appropriate host cell. For example, in certain embodiments, the TLR6 structural gene may be cloned into the commercially available mammalian expression vector pCI-neo. In this case, the TLR6 structural gene may be cloned into the vector's cloning region using the NheI and MluI restrictions sites. In such an embodiment, after transfection of the vector into a mammalian cell, the TLR6 structural gene is under the transcriptional control of the vector's CMV enhancer/promoter region.

The second nucleic acid sequence encodes a reporter that is capable of generating a detectable signal when expressed in a host cell under conditions appropriate for generating the desired detectable signal. A wide variety of suitable reporter systems are known. For example, luciferase gene expression may generate a detectable luminescent signal under appropriate conditions. As another example, β-galactosidase expression can generate a detectable color change under appropriate conditions. As yet another example, production and secretion of an immune system compound may be detected by an enzyme-linked immunosorbent assay (ELISA). These and other reporter systems are known and assays for generating the detectable signals are commercially available.

The second nucleic acid sequence is operably linked to a second expression control sequence that includes a promoter sequence selected to be inducible by activation of a TLR. Thus, expression and activation of a TLR, whether naturally expressed by the recombinant cell or encoded by the first nucleic acid sequence, will induce gene expression from the second expression control sequence, thereby causing expression of the reporter, which may be detected by performing an assay designed to detect expression of the reporter. The second expression control sequence may include any suitable nucleotide sequence that can induce expression (e.g., a promoter) of a structural gene upon activation of the TLR encoded by the first nucleic acid sequence. Nucleotide sequences suitable for use as second expression control sequences include promoter sequences of TLR-inducible genes including but not limited to genes encoding cytokines, chemokines, co-stimulatory markers, and defensins. In certain embodiments, the second expression control sequence includes an IFN-α1 promoter.

When the reporter system being employed to detect TLR activation includes detecting production and secretion of an immune system compound with an appropriate ELISA assay, the second expression control sequence may include the promoter of the gene encoding the immune system compounds being expressed and detected as the reporter. However, in certain embodiments, it may be desirable to express the immune system compound from a heterologous promoter.

When the gene expression system includes both a first nucleic acid sequence and a second nucleic acid sequence, the first nucleic acid sequence and the second nucleic acid sequence may be contained within a single vector. Alternatively, the first nucleic acid sequence and the second nucleic acid sequence may be on separate vectors and co-transfected into a suitable host cell. In certain embodiments, for example, the first nucleic acid sequence may be cloned into the pCI-neo vector as described above, while the second nucleic acid sequence can be cloned into a reporter vector. One example of a commercially available reporter vector is the pGL3-Enhancer vector, which includes a luciferase reporter gene downstream of a cloning site for cloning a promoter sequence of interest. In some embodiments, the promoter of a TLR-inducible immune system compound may be cloned into the pGL3-Enhancer cloning site. In one such embodiment, the IFN-α promoter may be cloned into the pGL3-Enhancer cloning site.

Suitable host cells include any transfectable cells capable of expressing exogenous mammalian genes. In some embodiments, the host cells may be mammalian cells such as human cells or mouse cells. For example, suitable host cells include human cells or descendants of a human cell including but not limited to Namalwa cells or HEK293 cells. Alternatively, the host cells may be mouse cells or descendants of a mouse cell including but not limited to RAW 264.7 cells.

In one embodiment, the host cells include Namalwa cells. Namalwa cells have certain characteristics that may be particularly desirable for certain embodiments of the present invention. For example, Namalwa cells can include an expressible chromosomal IFN-α gene locus. Thus, upon appropriate stimulation (e.g., viral infection), Namalwa cells can be induced to produce and secrete IFN-α from the chromosomal IFN-α gene locus. However, Namalwa cells do not naturally express certain TLRs (e.g., TLR6, TLR7, or TLR9). Certain agonists of such TLRs have been shown to induce IFN-α expression in other cell types (e.g., PMBCs), but may not induce IFN-α expression in Namalwa cells unless a functional level of TLR expression is provided.

Namalwa cells transfected with an appropriate gene expression system may be capable of expressing a functional level of the TLR provided by the expression system. Thus, Namalwa cells transfected with an appropriate expression system may inducibly express IFN-α as a result of activating the cloned TLR (e.g., by exposure of the transfected Namalwa cells to an agonist). Thus, certain transfected cell lines permit one to identify a TLR agonist using an assay that detects TLR-mediated IFN-α expression by Namalwa cells.

Namalwa cells transfected with certain expression systems can provide alternative means of detecting TLR activation and, therefore, alternate assays for identifying TLR agonists. First, Namalwa cells transfected with an appropriate expression system may generate a detectable signal as a result of TLR-mediated expression of the expression system reporter (see Table 2). Second, Namalwa cells transfected with an expression system that provides functional TLR activity may provide TLR-mediated IFN-α expression from the chromosomal IFN-α gene locus.

Assays

Assays according to the present invention may be performed using any suitable recombinant cell line. The recombinant cell line may be constructed by transfecting any suitable expression system into any suitable host cell. In the description of particular assays that follow, certain assay tools such as particular recombinant cell lines, particular gene expression systems, or particular host cells may be identified. However, many alternative assay tools may provide the features of the tools specifically identified and, consequently, may be suitable for use in assays according to the present invention. Such alternative embodiments are explicitly included in the scope of the present invention.

Also, each assay may or may not be performed in conjunction with one or more appropriate controls. Controls may be performed to assist in quantifying results or to ensure that the assay is performing as intended. However, with experience, one skilled in the art may develop sufficient familiarity with a particular assay that performing a control may not always be necessary to perform an assay of the present invention.

In some embodiments, assays according to the present invention may be designed to detect activation of a TLR. Such assays include providing a recombinant cell line having an appropriate gene expression system. Generally, an appropriate gene expression system includes a reporter that is (a) capable of generating a detectable signal when the reporter is expressed and the transfected cell is exposed to conditions that are appropriate for generating the detectable signal, and (b) operably linked to an expression control sequence that is capable of being induced by an activated TLR. The assays also include exposing the recombinant cell line to a TLR agonist, thereby activating the TLR and inducing expression of the reporter from the TLR-inducible expression control sequence; providing conditions appropriate for generating the reporter's detectable signal, thereby generating a detectable signal from the expressed reporter; and detecting the detectable signal, thereby detecting activation of the TLR.

In certain embodiments, the expression control sequence to which the reporter is operably linked may be a promoter of a TLR-inducible protein including but not limited to a cytokine, a chemokine, a co-stimulatory marker, or a defensin.

The recombinant cell line may be derived from a host cell that naturally expresses a functional level of one or more TLRs. In such embodiments, the gene expression system is not required to include a nucleic acid sequence that encodes a TLR. However, the gene expression system may include a nucleic acid sequence that encodes a TLR. For such assays, it may be desirable to measure any background level of detectable signal generated by the recombinant cell line before transfection with the nucleic acid sequence that encodes the TLR. In this way, one can obtain an indication of the extent of the detectable signal that is attributable to activation of the TLR expressed from the expression system if such an indication is desired.

When the gene expression system includes a nucleic acid sequence that encodes a TLR, one may select any TLR from any species for inclusion in the expression system. Accordingly, the nucleic acid sequence that encodes the TLR may include any one of the published TLR nucleotide sequences, any nucleotide sequence containing one or more degenerate variants of a published TLR nucleotide sequence, any nucleotide sequence that encodes a published TLR amino acid sequence, or any nucleotide sequence that encodes a protein having one or more conservative amino acid substitutions compared to a published TLR amino acid sequence.

In some embodiments in which the recombinant cell line includes a nucleic acid sequence encoding a TLR, a single vector may contain a first nucleic acid sequence that encodes the reporter and a second nucleic acid sequence that encodes the TLR. Alternatively, the first nucleic acid sequence and the second nucleic acid sequence may exist on separate vectors so that the host cells must be co-transfected with both vectors in order for the recombinant cell line to include entire gene expression system.

The gene expression system may include any suitable reporter operably linked to any suitable TLR-inducible expression control sequence. Suitable reporters are described in the detailed description of the gene expression system included in the description of assay tools provided above.

In one particular embodiment, the recombinant cell line is derived from the human lymphoblastoid Namalwa cell line. Namalwa cells lack a functional level of TLR6 activity. The recombinant cell line is obtained by co-transfecting Namalwa cells with two vectors that, together, provide a gene expression system: the first vector includes a nucleic acid sequence that encodes human TLR6 operably linked to an expression control sequence; the second vector contains a nucleic acid sequence that encodes a luciferase reporter gene that is operably linked to an IFN-α promoter. The IFN-α promoter is inducible by activation of TLR6. A culture of the recombinant cells is contacted with an agonist of TLR6, thereby activating TLR6 that has been expressed from the first vector of gene expression system. The activation of TLR6 induces expression from the IFN-α promoter on the second vector of the gene expression system. Expression from the IFN-α promoter results in expression of the luciferase reporter gene. The recombinant cells, which are now expressing the luciferase reporter, are contacted with a luciferase reagent that generates a luminescent signal when allowed to react with luciferase. Detection of the luminescent signal indicates expression of the luciferase reporter from the IFN-α promoter that, in turn, indicates activation of TLR6.

As indicated above in the detailed description of the assay tools, various suitable reporter systems may be used in alternative embodiments of assays according to the present invention. Also as indicated above, one feature of constructing the recombinant cell line from Namalwa host cells is the cells can produce and secrete IFN-α expressed from the chromosomal IFN-α gene locus of the Namalwa cell. Thus, detection of IFN-α production (e.g., by ELISA) may be used as a reporter of TLR activation. When used in conjunction with a reporter encoded by the gene expression system, the use of two independent reporters may provide certain embodiments of the assays of the present invention with an internal control.

In some alternative embodiments, assays according to the present invention may be designed to identify agonists of a particular TLR. Generally, such assays include providing a recombinant cell line constructed by transfecting host cells with a gene expression system that includes (a) a first nucleic acid sequence that encodes a particular TLR, and (b) a second nucleic acid sequence that encodes a reporter operably linked to an expression control sequence that is inducible by activation of the TLR encoded by the expression system. The assays also include contacting cell cultures of the recombinant cell line with one or more test compounds, and then exposing the cell cultures to conditions effective for generating a detectable signal from the reporter in the event that the reporter is expressed. Detection of a TLR-mediated detectable signal indicates that expression of the reporter is at least partially attributable to activation of the TLR by the test compound, thereby identifying the test compound as an agonist of the TLR.

As with the assays described above that are designed for detecting TLR activation, assays for detecting TLR agonists include a gene expression system that may include one or more vectors, a nucleic acid sequence that encodes any suitable reporter, and any suitable TLR-inducible expression control sequence. Furthermore, the gene expression system can include a nucleic acid sequence that encodes any particular TLR. Thus, an assay may be designed to identify agonists of any particular TLR.

Detection of a TLR-mediated detectable signal may include a determination of background detectable signal generated by the recombinant cell line prior to transfection with a nucleic acid sequence that encodes a particular TLR. A recombinant cell line may, in some embodiments, naturally possess a certain level of TLR expression that can induce expression of the reporter, thereby generating background signal. Alternatively, background expression of the reporter may result from induction of the expression control sequence that regulates expression of the reporter coming from an alternative (i.e., non-TLR) source. Once a background level of detectable signal is determined for the recombinant cell line, it may not be necessary to determine the background signal generation every time the assay is performed.

In one particular embodiment, the recombinant cell line includes Namalwa cells, cells that lack a functional level of natural TLR6 expression. The recombinant cell line is constructed by co-transfecting Namalwa cells with a gene expression system that includes two vectors: a first vector that includes a first nucleic acid sequence that encodes human TLR6 operably linked to an expression control sequence; and a second vector that includes a second nucleic acid sequence that encodes a luciferase reporter operably linked to an IFN-α promoter. The first nucleic acid sequence permits the recombinant cells to functionally express TLR6. The second nucleic acid sequence allows one to detect activation of the TLR6 expressed from the first nucleic acid sequence.

In this particular embodiment, a culture of the recombinant cells is dispensed into wells of a multi-well test plate. A different test compound is added to each well. A test compound that acts as a TLR6 agonist will activate the TLR6 expressed from the first vector of the gene expression system, thereby inducing expression from the IFN-α promoter operably linked to the luciferase reporter on the second vector of the gene expression system. The recombinant cells, which are now expressing the luciferase reporter, are contacted with a luciferase reagent that generates a TLR-mediated detectable signal only when the luciferase reporter is expressed. Detection of a TLR-mediated detectable signal in a particular well of the multi-well plate indicates expression of the luciferase reporter from the IFN-α promoter that, in turn, indicates activation of TLR6 by the test compound added to the recombinant cells in that well. A test compound that activates TLR6 is an agonist of TLR6.

Test compounds may be added to wells containing recombinant cells in any manner appropriate for the design of a particular assay. For example, the same test compound may be added to each of a plurality of wells, thereby generating multiple data points for that test compound. Alternatively, a different test compound may be added to each well. In this way, the number of test compounds that can be screened in a single assay can be maximized. In some embodiments, test compound may even be omitted from a certain number of wells, e.g., in order to generate one or more controls.

In another particular embodiment, the assay may be designed to identify agonists of TLR7 by designing the recombinant cell line to include a gene expression system that includes a nucleic acid sequence that encodes human TLR7. In all other respects, the assay may be performed as described above for the detection of TLR6 agonists.

Additional alternative embodiments include assays that are designed to identify agonists of any one of the human TLRs or any non-human TLR merely by designing the gene expression system to include a nucleic acid sequence that encodes the desired TLR.

The present invention also provides TLR agonist compounds identified using an assay according to certain embodiments of the present invention. As described above, the expression systems and recombinant cell lines may provide the ability to design assays that can identify TLR agonists that are not detectable using previously known TLR activation assays. The TLR agonists may include chemical structures similar in certain respects to the chemical structures of known IRM compounds. Alternatively, assays according to the present invention may be used for screening (e.g., high throughput screening) chemically diverse compounds that may lead to the discovery of new TLR agonists, some of which may contain new chemical core structures capable of activating TLRs.

The present invention also provides pharmaceutical compositions containing a TLR agonist identified using an assay according to the present invention, or a pharmaceutically acceptable salt thereof, in an amount effective for inducing a TLR-mediated cellular response.

In still other embodiments, assays according to the present invention may be designed to identify antagonists of a particular TLR. Generally, an assay may be designed to identify an antagonist of a particular TLR by designing the recombinant cell line to include a gene expression system having (a) a first nucleic acid sequence that encodes a particular TLR, and (b) a second nucleic acid sequence that encodes a reporter operably linked to a TLR-inducible expression control sequence. Aliquots of the recombinant cell line may be dispensed into wells of a multi-well test plate. A different test compound can be added to each well, and then a known agonist of the particular TLR can be added to each well. In such assays, the agonist of the particular TLR will induce expression of the reporter and generation of a detectable signal unless the test compound acts as an antagonist of the particular TLR. Therefore, antagonists of the particular TLR can be identified by detecting wells exhibiting something less than a baseline TLR-mediated detectable signal.

The baseline TLR-mediated detectable signal may be determined by use of a control when performing the assay. However, with experience, one skilled in the art may develop sufficient familiarity with a particular assay that explicit use of controls may not always be necessary to identify a TLR antagonist using the methods of the present invention.

As with the assays described above that are designed for identifying TLR agonists, assays for detecting TLR antagonists include a gene expression system that may include one or more vectors, a nucleic acid sequence that encodes any suitable reporter, any suitable TLR-inducible expression control sequence, and a nucleic acid sequence that encodes any particular TLR. Thus, an assay may be designed to identify antagonists of any particular TLR.

In one particular embodiment, an assay that identifies antagonists of human TLR6 may be designed using the recombinant cell line described above for the identification of TLR6 agonists. The recombinant cells are dispensed into the wells of a multi-well test plate. A different test compound is added to each well. A known TLR6 agonist such as any one of the IRM compounds listed in Table 1 can be added to each well.

Generation and detection of the TLR-mediated detectable signal can be performed as described above for assays designed to detect TLR activation or identify TLR agonists. The TLR-mediated detectable signal from each well can be compared to a baseline TLR-mediated detectable signal. As noted above, the baseline detectable signal may be determined, for example, from experience or by performing a positive control. Test compounds that inhibit the TLR-mediate detectable signal compared to the baseline detectable signal can be identified as antagonists of TLR6.

In alternative embodiments, test compounds may be added to the wells in any desired manner, as described above with regard to assays designed to identify TLR agonists.

Other alternative embodiments include assays designed to identify antagonists of any one of the human TLRs or any non-human TLR. Such alternative embodiments may be performed by designing the gene expression system to include a nucleic acid sequence that encodes the desired TLR.

The present invention also provides TLR antagonist compounds identified using an assay according to certain embodiments of the present invention. As described above, the expression systems and recombinant cell lines may provide the ability to design assays that can identify TLR antagonists that are not detectable using previously known TLR activation assays. The TLR antagonists may include chemical structures similar in certain respects to the chemical structures of known IRM compounds. Alternatively, assays according to the present invention may be used for screening (e.g., high throughput screening) chemically diverse compounds that may lead to the discovery of new TLR antagonists, some of which may contain new chemical core structures capable of activating TLRs.

The present invention also provides pharmaceutical compositions containing a TLR antagonist identified using an assay according to the present invention, or a pharmaceutically acceptable salt thereof, in an amount effective for inhibiting a TLR-mediated cellular response.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

Construction of Vectors

The vector pIFN-α1-luc was constructed by inserting BglII sites at both ends of the human IFN-α1 promoter (SEQ ID NO:21). The BglII sites were inserted into the IFN-α1 promoter and the sequence was amplified using the primer pair of SEQ ID NO:22 and SEQ ID NO:23. The amplified IFN-α1 promoter was cloned into the pGL3-Enhancing vector (Promega Corp., Madison, Wis.) at the BglII site.

The vector pCI-TLR6 was constructed by inserting SEQ ID NO:11 (GenBank Accession No. NM 006068), which includes the human TLR6 coding sequence, into the pCI-neo mammalian expression vector (Promega Corp.) at the vector's NheI and MluI restriction sites.

Transfections

Unless otherwise indicated, all incubations were performed at 37° C. with 5% CO₂ at 98% humidity.

Culture medium was prepared from complete RPMI 1640 medium (BioSource International, Inc., Camarillo, Calif.). Fetal bovine serum (Atlas Biologicals, Inc., Ft. Collins, Colo.) was added to a final concentration of 7.5% (vol/vol); L-glutamine (BioSource International, Inc.) was added to 5 mM; and sodium pyruvate (BioSource International, Inc.) was added to 1 mM.

Burkitt's Lymphoma lymphoblastoid Namalwa cells (ATCC Accession No. CRL-1432) were grown by incubation in culture medium overnight. Cells were harvested by centrifugation in a tabletop centrifuge (1200 RPM for 5 minutes), and then resuspended in phosphate buffered sucrose to a concentration of 1.3×10⁷ cells per milliliter.

For each transfection, a 750 μL aliquot of the cell suspension was placed in an electroporation cuvette with 4 mm gaps. 10 μg of the pIFN-α1-luc vector and 10 μg of the pCI-TLR6 vector were added to the electroporation cuvette. The cell and vector mixtures were incubated at room temperature for 5 minutes. The cells were electroporated using a BioRad Gene Pulser (BioRad Laboratories, Hercules, Calif.) set to at 500 μF capacitance and 0.27 volts, then incubated at room temperature for 5 minutes.

The electroporated cells were suspended in 10 mLs of culture medium and incubated overnight. Dead cells and debris were removed after 24 hours using a MACS Dead Cell Removal kit (Miltenyi Biotec, Auburn, Calif.). Cells were resuspended in 10 mLs of culture medium and incubated for an additional 24 hours.

Transfected cells were selected by adding G418 (Promega Corp., Madison, Wis.) to a final concentration of 1 mg/mL and incubating the cells for seven days.

Assays

The selected transfected cells were counted and resuspended to a concentration of 1×10⁶ cell per mL in culture medium. 100 μl aliquots of cells were placed in the wells of a white-walled, white-bottomed 96-well plate (Corning, Inc. Corning, N.Y.). 1.0 μL of an IRM compound from Table 1 (prepared at 1 mM in 100% DMSO) was added to some cell aliquots so that the final concentration of IRM compound was 10 μM. As a positive control, some cell aliquots were incubated with Sendai virus instead of IRM compound. As a negative control, some cell aliquots were incubated with DMSO without IRM compound. In all cases, the cells were incubated for 18 hours.

TABLE 1
IRM Compounds

| Compound | Chemical Name | Citation |
|---|---|---|
| IRM 1 | 4-amino-2-ethoxymethyl-α,α- | U.S. Pat. No. |
| | dimethyl-6,7,8,9-tetrahydro-1H- | 5,352,784 |
| | imidazo[4,5-c]quinoline-1- | Example 91 |
| | ethanol | |
| IRM 2 | 4-amino-α,α,2-trimethyl- | U.S. Pat. No. |
| | 1H-imidazo[4,5-c]quinoline- | 5,266,575 |
| | 1-ethanol | Example C1 |
| IRM 3 | N-[4-(4-amino-2-butyl-1H- | U.S. Pat. No. |
| | imidazo[4,5-c]quinolin-1- | 6,331,539 |
| | yl)butyl]methanesulfonamide | Example 6 |
| IRM 4 | 1-{2-[3-(3-pyridyl)- | WO 02/46193 |
| | propoxy]ethyl}-1H-imidazo[4, | Example 33 |
| | 5-c]quinolin-4-amine | |
| IRM 5 | 2-butyl-1-(2-methylpropyl)-1H- | U.S. Pat. No. |
| | imidazo[4,5-c][1, | 6,194,425 |
| | 5]naphthyridin-4-amine | Example 39 |
| IRM 6 | 2-butyl-6,7,8,9-tetrahydro-1- | U.S. Pat. No. |
| | (2-methylpropyl)-1H-imidazo[4, | 6,194,425 |
| | 5-c][1,5]naphthyridin-4-amine | Example 40 |
| IRM 7 | N³-{4-[4-amino-2-(2- | U.S. Pat. No. |
| | methoxyethyl)-1H-imidazo[4,5- | 6,451,810 |
| | c]quinolin-1-yl]butyl}-6-(1H- | Example 60 |
| | 1-pyrrolyl)nicotinamide | |
| IRM 8 | 2-ethyl-1-[5-(methylsulfonyl)- | WO 02/46192 |
| | pentyl]-1H-imidazo[4,5- | Example 13 |
| | c]quinolin-4-amine | |

The plates were equilibrated to room temperature before 1 volume of reconstituted LucLight Plus (Packard Instruments, Meriden, Conn.) was added to each aliquot of cells. Each well of the plate was read on an LJL Analyst (LJL Biosystems, Inc., Sunnyvale, Calif.) set with a 5 minute dark adapt. Data from a representative experiment are shown in Table 2. The data are expressed as the fold increase in luciferase induction off of the IFN-α1 promoter in cell aliquots incubated with the indicated stimulant compared to the negative control in which the cell aliquots were incubated with only DMSO.

TABLE 2
TLR Expression by pIFN-α1-luc/pCI-TLR6
Co-Transfected Namalwa cells

| | Fold Increase in |
|---|---|
| Stimulant | Luciferase Induction |
| IRM1 | 3.6 |
| IRM2 | 2.7 |
| IRM3 | 2.6 |
| IRM4 | 4.0 |
| IRM5 | 3.2 |
| IRM6 | 2.9 |
| IRM7 | 3.2 |
| IRM8 | 2.3 |
| Sendai virus | 2.7 |

The complete disclosures of the patents, patent documents, and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. A method of detecting activation of a TLR in a cell comprising:
providing a cell culture comprising cells transfected with a nucleic acid sequence that encodes a reporter that (a) generates a detectable signal when the reporter is expressed and the cell is exposed to conditions effective for generating the detectable signal, and (b) is operably linked to an expression control sequence that is induced by activation of a TLR and comprises a cytokine promoter, a chemokine promoter, a co-stimulatory marker promoter, or a defensin promoter;
exposing the cell culture to a compound that activates a TLR;
providing conditions effective for generating the detectable signal; and
detecting the detectable signal.

2. The method of claim 1 wherein the expression control sequence comprises an IFN-α promoter.

3. The method of claim 1 wherein the detectable signal comprises luciferase activity, β-galactosidase activity, or a positive signal from an enzyme-linked immunosorbent assay.

4. The method of claim 1 wherein the cell culture comprises mammalian cells or descendents of a mammalian cell.

5. The culture cell of claim 4 wherein the cell culture comprises human cells or descendents of a human cell.

6. The method of claim 1 wherein the cells are further transfected with a second nucleic acid sequence that encodes a TLR operably linked to a second expression control sequence.

7. The method of claim 6 wherein the first nucleic acid sequence comprises the nucleotide sequence of SEQ ID NO:1, SEQ ID NO:3, SEQ ID NO:5, SEQ ID NO:7, SEQ ID NO:9, SEQ ID NO:1, SEQ ID NO:13, SEQ ID NO:15, SEQ ID NO:17, SEQ ID NO:19, or a degenerate variant of any of the foregoing.

8. The method of claim 6 wherein the first nucleic acid sequence comprises a nucleotide sequence that encodes a polypeptide having the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, or any one of the foregoing sequences with one or more conservative amino acid substitutions.

9. The method of claim 6 wherein the nucleic acid sequence that encodes the reporter and the second nucleic acid sequence are contained on a single vector.

10. The method of claim 6 wherein the nucleic acid sequence that encodes the reporter is contained on a first vector and the second nucleic acid sequence is contained on a second vector.

11. A method of identifying a TLR agonist-comprising:
providing a cell culture comprising cells transfected with:
a first nucleic acid sequence that comprises a nucleotide sequence that encodes a TLR operably linked to a first expression control sequence and
a second nucleic acid sequence that encodes a reporter that (a) generates a detectable signal when the reporter is expressed and the transfected cell is exposed to conditions effective for generating the detectable signal, and (b) is operably linked to a second expression control sequence that is induced by activation of a TLR;
contacting the cell culture with a test compound;
providing conditions effective for generating the detectable signal, thereby generating a TLR-mediated detectable signal if the TLR has been activated; and
identifying the compound as an agonist of the TLR if a TLR-mediated detectable signal is detected.

12. The method of claim 11 wherein the first nucleic acid sequence comprises the nucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, or a degenerate variant of any of the foregoing.

13. The method of claim 11 wherein the first nucleic acid sequence comprises a nucleotide sequence that encodes a polypeptide having the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, or any one of the foregoing sequences with one or more conservative amino acid substitutions.

14. The method of claim 11 wherein the second expression control sequence comprises an IFN-α promoter.

15. The method of claim 11 wherein the detectable signal comprises luciferase activity, β-galactosidase activity, or a positive signal from an enzyme-linked immunosorbent assay.

16. The method of claim 11 wherein the cell culture comprises mammalian cells or descendents of a mammalian cell.

17. The method of claim 16 wherein the cell culture comprises human cells or descendents of a human cell.

18. The method of claim 11 wherein the first nucleic acid sequence and the second nucleic acid sequence are included in a single vector.

19. The method of claim 11 wherein the first nucleic acid sequence and the second nucleic acid sequence are located on separate vectors.

20. The method of claim 19 wherein the cell culture comprises cells co-transfected with the separate vectors.

21. The method of claim 11 wherein the cell culture comprises cells that, prior to transfection with the first nucleic acid sequence, exhibit no detectable function of the Toll-like receptor encoded by the first nucleic acid sequence.

22. The method of claim 11 wherein the second expression control sequence comprises a cytokine promoter, a chemokine promoter, a co-stimulatory marker promoter, or a defensin promoter

23. A TLR agonist identified by the method of claim 11.

24. A pharmaceutical composition comprising a TLR agonist identified by the method of claim 23 or a pharmaceutically acceptable salt thereof.

25. A method of identifying an antagonist of a TLR comprising:
providing a cell culture that comprises cells transfected with:
a first nucleic acid sequence that comprises a nucleotide sequence that encodes the TLR operably linked to a first expression control sequence, and
a second nucleic acid sequence that encodes a reporter that (a) is operably linked to a second expression control sequence that is induced by activation of the TLR, and (b) generates a detectable signal when the reporter is expressed and the transfected cell is exposed to conditions effective for generating the detectable signal;
contacting the cell culture with an agonist of the TLR, thereby permitting generation of a detectable signal under conditions effective for generating a detectable signal;
contacting the cell culture with a test compound;
providing conditions effective for generating the detectable signal;
measuring the detectable signal; and
identifying the compound as an antagonist of the TLR if the detectable signal is less than a baseline TLR-mediated detectable signal.

26. A TLR antagonist identified by the method of claim 25.

27. A pharmaceutical composition comprising a TLR antagonist identified by the method of claim 26 or a pharmaceutically acceptable salt thereof.


SEQUENCE LISTING (23 sequences)

SEQ ID NO: 1 - DNA, 2832 bp, Homo sapiens
acagactgccaaatggaacagacaagcaggttgtcttgtgttaaagaaaatgagatatgagtcagttactcccggaggcaatgctgctgttcagctcttgtgtttttgtggccagggtcttcatgaacactaataggggtaccaggccctcttccttgttagaagaaatcaggataacaaaggtatattgggcacccctacaaaaggaatctgtatctgtatcaagatgatctgaagaacagcttctacctttaggaatgtctagtgttccaaaatgactagcatcttccattttgccattatcttcatgttaatacttcagatcagaatacaattatctgaagaaagtgaatttttagttgataggtcaaaaaacggtctcatccacgttcctaaagacctatcccagaaaacaacaatcttaaatatatcgcaaaattatatatctgagctttggacttctgacatcttatcactgtcaaaactgaggattttgataatttctcataatagaatccagtatcttgatatcagtgttttcaaattcaaccaggaattggaatacttggatttgtcccacaacaagttggtgaagatttcttgccaccctactgtgaacctcaagcacttggacctgtcatttaatgcatttgatgccctgcctatatgcaaagagtttggcaatatgtctcaactaaaatttctggggttgagcaccacacacttagaaaaatctagtgtgctgccaattgctcatttgaatatcagcaaggtcttgctggtcttaggagagacttatggggaaaaagaagaccctgagggccttcaagactttaacactgagagtctgcacattgtgttccccacaaacaaagaattccattttattttggatgtgtcagtcaagactgtagcaaatctggaactatctaatatcaaatgtgtgctagaagataacaaatgttcttacttcctaagtattctggcgaaacttcaaacaaatccaaagttatcaagtcttaccttaaacaacattgaaacaacttggaattctttcattaggatcctccagctggtttggcatacaactgtatggtatttctcaatttcaaacgtgaagctacagggtcagctggacttcagagattttgattattctggcacttccttgaaggccttgtctatacaccaagttgtcagcgatgtgttcggttttccgcaaagttatatctatgaaatcttttcgaatatgaacatcaaaaatttcacagtgtctggtacacgcatggtccacatgctttgcccatccaaaattagcccgttcctgcatttggatttttccaataatctcttaacagacacggtttttgaaaattgtgggcaccttactgagttggagacacttattttacaaatgaatcaattaaaagaactttcaaaaatagctgaaatgactacacagatgaagtctctgcaacaattggatattagccagaattctgtaagctatgatgaaaagaaaggagactgttcttggactaaaagtttattaagtttaaatatgtcttcaaatatacttactgacactattttcagatgtttacctcccaggatcaaggtacttgatcttcacagcaataaaataaagagcattcctaaacaagtcgtaaaactggaagctttgcaagaactcaatgttgctttcaattctttaactgaccttcctggatgtggcagctttagcagcctttctgtattgatcattgatcacaattcagtttcccacccatcagctgatttcttccagagctgccagaagatgaggtcaataaaagcaggggacaatccattccaatgtacctgtgagctaggagaatttgtcaaaaatatagaccaagtatcaagtgaagtgttagagggctggcctgattcttataagtgtgactacccggaaagttatagaggaaccctactaaaggactttcacatgtctgaattatcctgcaacataactctgctgatcgtcaccatcgttgccaccatgctggtgttggctgtgactgtgacctccctctgcatctacttggatctgccctggtatctcaggatggtgtgccagtggacccagacccggcgcagggccaggaacatacccttagaagaactccaaagaaatctccagtttcatgcatttatttcatatagtgggcacgattctttctgggtgaagaatgaattattgccaaacctagagaaagaaggtatgcagatttgccttcatgagagaaactttgttcctggcaagagcattgtggaaaatatcatcacctgcattgagaagagttacaagtccatctttgttttgtctcccaactttgtccagagtgaatggtgccattatgaactctactttgcccatcacaatctctttcatgaaggatctaatagcttaatcctgatcttgctggaacccattccgcagtactccattcctagcagttatcacaagctcaaaagtctcatggccaggaggacttatttggaatggcccaaggaaaagagcaaacgtggccttttttgggctaacttaagggcagccattaatattaagctgacagagcaagcaaagaaatagattacacatcaagtgaaaaatattcctcctgttgatattgctgcttttggaagttccaacaatgactttattttgcatcagcatagatgtaaacacaattgtgagtgtatgatgtaggtaaaaatatataccttcgggtcgcagttcaccatttatatgtggtattaaaaattaatgaaatgatataactttgattt

SEQ ID NO: 2 - protein, 786 aa, Homo sapiens
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSSLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCIYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 3 - DNA, 2621 bp, Homo sapiens
cagtgtttggtgttgcaagcaggatccaaaggagacctatagtgactcccaggagctcttagtgaccaagtgaaggtacctgtggggctcattgtgcccattgctctttcactgctttcaactggtagttgtgggttgaagcactggacaatgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctaggttcccatatttaagaccagtctttgtctagttgggatctttatgtcactagttatagttaagttcattcagacataattatataaaaactacgtggatgtaccgtcatttgagga

SEQ ID NO: 4 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 5 - DNA, 3057 bp, Homo sapiens
cactttcgagagtgccgtctatttgccacacacttccctgatgaaatgtctggatttggactaaagaaaaaaggaaaggctagcagtcatccaacagaatcatgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtacattaaatttatttaaatattcaattagcaaaggagaaactttctcaatttaaaaagttctatggcaaatttaagttttccataaaggtgttataatttgtttattcatatttgtaaatgattatattctatcacaattacatctcttctaggaaaatgtgtctccttatttcaggcctatttttgacaattgacttaattttacccaaaataaaacatataagcacgtaaaaaaaaaaaaaaaaaa

SEQ ID NO: 6 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 7 - DNA, 3811 bp, Homo sapiens
acagggccactgctgctcacagaagcagtgaggatgatgccaggatgatgtctgcctcgcgcctggctgggactctgatcccagccatggccttcctctcctgcgtgagaccagaaagctgggagccctgcgtggagacttggccctaaaccacacagaagagctggcatgaaacccagagctttcagactccggagcctcagcccttcaccccgattccattgcttcttgctaaatgctgccgttttatcacggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctgaagaggaaaaataaaaacctcctgaggcatttcttgcccagctgggtccaacacttgttcagttaataagtattaaatgctgccacatgtcaggccttatgctaagggtgagtaattccatggtgcactagatatgcagggctgctaatctcaaggagcttccagtgcagagggaataaatgctagactaaaatacagagtcttccaggtgggcatttcaaccaactcagtcaaggaacccatgacaaagaaagtcatttcaactcttacctcatcaagttgaataaagacagagaaaacagaaagagacattgttcttttcctgagtcttttgaatggaaattgtattatgttatagccatcataaaaccattttggtagttttgactgaactgggtgttcactttttcctttttgattgaatacaatttaaattctacttgatgactgcagtcgtcaaggggctcctgatgcaagatgccccttccattttaagtctgtctccttacagaggttaaagtctaatggctaattcctaaggaaacctgattaacacatgctcacaaccatcctggtcattctcgaacatgttctattttttaactaatcacccctgatatatttttatttttatatatccagttttcatttttttacgtcttgcctataagctaatatcataaataaggttgtttaagacgtgcttcaaatatccatattaaccactatttttcaaggaagtatggaaaagtacactctgtcactttgtcactcgatgtcattccaaagttattgcctactaagtaatgactgtcatgaaagcagcattgaaataatttgtttaaagggggcactcttttaaacgggaagaaaatttccgcttcctggtcttatcatggacaatttgggctataggcatgaaggaagtgggattacctcaggaagtcaccttttcttgattccagaaacatatgggctgataaacccggggtgacctcatgaaatgagttgcagcagatgtttatttttttcagaacaagtgatgtttgatggacctatgaatctatttagggagacacagatggctgggatccctcccctgtacccttctcactgacaggagaacta

SEQ ID NO: 8 - protein, 799 aa, Homo sapiens
MELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 9 - DNA, 1261 bp, Homo sapiens
tgttgggatgtttttgagggactttctcatcttcaagttctgtatttgaatcataactatcttaattcccttccaccaggagtatttagccatctgactgcattaaggggactaagcctcaactccaacaggctgacagttctttctcacaatgatttacctgctaatttagagatcctggacatatccaggaaccagctcctagctcctaatcctgatgtatttgtatcacttagtgtcttggatataactcataacaagttcatttgtgaatgtgaacttagcacttttatcaattggcttaatcacaccaatgtcactatagctgggcctcctgcagacatatattgtgtgtaccctgactcgttctctggggtttccctcttctctctttccacggaaggttgtgatgaagaggaagtcttaaagtccctaaagttctcccttttcattgtatgcactgtcactctgactctgttcctcatgaccatcctcacagtcacaaagttccggggcttctgttttatctgttataagacagcccagagactggtgttcaaggaccatccccagggcacagaacctgatatgtacaaatatgatgcctatttgtgcttcagcagcaaagacttcacatgggtgcagaatgctttgctcaaacacctggacactcaatacagtgaccaaaacagattcaacctgtgctttgaagaaagagactttgtcccaggagaaaaccgcattgccaatatccaggatgccatctggaacagtagaaagatcgtttgtcttgtgagcagacacttccttagagatggctggtgccttgaagccttcagttatgcccagggcaggtgcttatctgaccttaacagtgctctcatcatggtggtggttgggtccttgtcccagtaccagttgatgaaacatcaatccatcagaggctttgtacagaaacagcagtatttgaggtggcctgaggatctccaggatgttggctggtttcttcataaactctctcaacagatactaaagaaagaaaaagaaaagaagaaagacaataacattccgttgcaaactgtagcaaccatctcctaatcaaaggagcaatttccaacttatctcaagccacaaataactcttcactttgtatttgcaccaagttatcattttggggtcctctctggaggttttttttttctttttgctactatgaaaacaacataaatctctcaattttcgtatcaaaaaaaaaaaaaaa

SEQ ID NO: 10 - protein, 204 aa, Homo sapiens [sequence omitted]

SEQ ID NO: 11 - DNA, 2753 bp, Homo sapiens
agaatttggactcatatcaagatgctctgaagaagaacaaccctttaggatagccactgcaacatcatgaccaaagacaaagaacctattgttaaaagcttccattttgtttgccttatgatcataatagttggaaccagaatccagttctccgacggaaatgaatttgcagtagacaagtcaaaaagaggtcttattcatgttccaaaagacctaccgctgaaaaccaaagtcttagatatgtctcagaactacatcgctgagcttcaggtctctgacatgagctttctatcagagttgacagttttgagactttcccataacagaatccagctacttgatttaagtgttttcaagttcaaccaggatttagaatatttggatttatctcataatcagttgcaaaagatatcctgccatcctattgtgagtttcaggcatttagatctctcattcaatgatttcaaggccctgcccatctgtaaggaatttggcaacttatcacaactgaatttcttgggattgagtgctatgaagctgcaaaaattagatttgctgccaattgctcacttgcatctaagttatatccttctggatttaagaaattattatataaaagaaaatgagacagaaagtctacaaattctgaatgcaaaaacccttcaccttgtttttcacccaactagtttattcgctatccaagtgaacatatcagttaatactttagggtgcttacaactgactaatattaaattgaatgatgacaactgtcaagttttcattaaatttttatcagaactcaccagaggttcaaccttactgaattttaccctcaaccacatagaaacgacttggaaatgcctggtcagagtctttcaatttctttggcccaaacctgtggaatatctcaatatttacaatttaacaataattgaaagcattcgtgaagaagattttacttattctaaaacgacattgaaagcattgacaatagaacatatcacgaaccaagtttttctgttttcacagacagctttgtacaccgtgttttctgagatgaacattatgatgttaaccatttcagatacaccttttatacacatgctgtgtcctcatgcaccaagcacattcaagtttttgaactttacccagaacgttttcacagatagtatttttgaaaaatgttccacgttagttaaattggagacacttatcttacaaaaaaatggattaaaagaccttttcaaagtaggtctcatgacgaaggatatgccttctttggaaatactggatgttagctggaattctttggaatctggtagacataaagaaaactgcacttgggttgagagtatagtggtgttaaatttgtcttcaaatatgcttactgactctgttttcagatgtttacctcccaggatcaaggtacttgatcttcacagcaataaaataaagagcgttcctaaacaagtcgtaaaactggaagctttgcaagaactcaatgttgctttcaattctttaactgaccttcctggatgtggcagctttagcagcctttctgtattgatcattgatcacaattcagtttcccacccatcggctgatttcttccagagctgccagaagatgaggtcaataaaagcaggggacaatccattccaatgtacctgtgagctaagagaatttgtcaaaaatatagaccaagtatcaagtgaagtgttagagggctggcctgattcttataagtgtgactacccagaaagttatagaggaagcccactaaaggactttcacatgtctgaattatcctgcaacataactctgctgatcgtcaccatcggtgccaccatgctggtgttggctgtgactgtgacctccctctgcatctacttggatctgccctggtatctcaggatggtgtgccagtggacccagactcggcgcagggccaggaacatacccttagaagaactccaaagaaacctccagtttcatgcttttatttcatatagtgaacatgattctgcctgggtgaaaagtgaattggtaccttacctagaaaaagaagatatacagatttgtcttcatgagaggaactttgtccctggcaagagcattgtggaaaatatcatcaactgcattgagaagagttacaagtccatctttgttttgtctcccaactttgtccagagtgagtggtgccattacgaactctattttgcccatcacaatctctttcatgaaggatctaataacttaatcctcatcttactggaacccattccacagaacagcattcccaacaagtaccacaagctgaaggctctcatgacgcagcggacttatttgcagtggcccaaggagaaaagcaaacgtgggctcttttgggctaacattagagccgcttttaatatgaaattaacactagtcactgaaaacaatgatgtgaaatcttaaaaaaatttaggaaattcaacttaagaaaccattatttacttggatgatggtgaatagtacagtcgtaagtaactgtctggaggtgcctccattatcctcatgccttcaggaaagacttaacaaaaacaatgtttcatctggggaactgagctaggcggtgaggttagcctgccagttagagacagcccagtctcttctggtttaatcattatgtttcaaattgaaacagtctcttttgagtaaatgctcagtttttcagctcctctccactctgctttcccaaatggattctgttg

SEQ ID NO: 12 - protein, 796 aa, Homo sapiens
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGSTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 13 - DNA, 5007 bp, Homo sapiens
actccagatataggatcactccatgccatcaagaaagttgatgctattgggcccatctcaagctgatcttggcacctctcatgctctgctctcttcaaccagacctctacattccattttggaagaagactaaaaatggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagcccttctttgcaaaacacaactgcctagtttaccaaggagaggcctggctgtttaaattgttttcatatatatcacaccaaaagcgtgttttgaaattcttcaagaaatgagattgcccatatttcaggggagccaccaacgtctgtcacaggagttggaaagatggggtttatataatgcatcaagtcttctttcttatctctctgtgtctctatttgcacttgagtctctcacctcagctcctgtaaaagagtggcaagtaaaaaacatggggctctgattctcctgtaattgtgataattaaatatacacacaatcatgacattgagaagaactgcatttctacccttaaaaagtactggtatatacagaaatagggttaaaaaaaactcaagctctctctatatgagaccaaaatgtactagagttagtttagtgaaataaaaaaccagtcagctggccgggcatggtggctcatgcttgtaatcccagcactttgggaggccgaggcaggtggatcacgaggtcaggagtttgagaccagtctggccaacatggtgaaaccccgtctgtactaaaaatacaaaaattagctgggcgtggtggtgggtgcctgtaatcccagctacttgggaggctgaggcaggagaatcgcttgaacccgggaggtggaggtggcagtgagccgagatcacgccactgcaatgcagcccgggcaacagagctagactgtctcaaaagaacaaaaaaaaaaaaacacaaaaaaactcagtcagcttcttaaccaattgcttccgtgtcatccagggccccattctgtgcagattgagtgtgggcaccacacaggtggttgctgcttcagtgcttcctgctctttttccttgggcctgcttctgggttccatagggaaacagtaagaaagaaagacacatccttaccataaatgcatatggtccacctacaaatagaaaaatatttaaatgatctgcctttatacaaagtgatattctctacctttgataatttacctgcttaaatgtttttatctgcactgcaaagtactgtatccaaagtaaaatttcctcatccaatatctttcaaactgttttgttaactaatgccatatatttgtaagtatctgcacacttgatacagcaacgttagatggttttgatggtaaaccctaaaggaggactccaagagtgtgtatttatttatagttttatcagagatgacaattatttgaatgccaattatatggattcctttcattttttgctggaggatgggagaagaaaccaaagtttatagaccttcacattgagaaagcttcagttttgaacttcagctatcagattcaaaaacaacagaaagaaccaagacattcttaagatgcctgtactttcagctgggtataaattcatgagttcaaagattgaaacctgaccaatttgctttatttcatggaagaagtgatctacaaaggtgtttgtgccatttggaaaacagcgtgcatgtgttcaagccttagattggcgatgtcgtattttcctcacgtgtggcaatgccaaaggctttactttacctgtgagtacacactatatgaattatttccaacgtacatttaatcaataagggtcacaaattcccaaatcaatctctggaataaatagagaggtaattaaattgctggagccaactatttcacaacttctgtaagc

SEQ ID NO: 14 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 15 - DNA, 3311 bp, Homo sapiens
ttctgcgctgctgcaagttacggaatgaaaaattagaacaacagaaacatggaaaacatgttccttcagtcgtcaatgctgacctgcattttcctgctaatatctggttcctgtgagttatgcgccgaagaaaatttttctagaagctatccttgtgatgagaaaaagcaaaatgactcagttattgcagagtgcagcaatcgtcgactacaggaagttccccaaacggtgggcaaatatgtgacagaactagacctgtctgataatttcatcacacacataacgaatgaatcatttcaagggctgcaaaatctcactaaaataaatctaaaccacaaccccaatgtacagcaccagaacggaaatcccggtatacaatcaaatggcttgaatatcacagacggggcattcctcaacctaaaaaacctaagggagttactgcttgaagacaaccagttaccccaaataccctctggtttgccagagtctttgacagaacttagtctaattcaaaacaatatatacaacataactaaagagggcatttcaagacttataaacttgaaaaatctctatttggcctggaactgctattttaacaaagtttgcgagaaaactaacatagaagatggagtatttgaaacgctgacaaatttggagttgctatcactatctttcaattctctttcacacgtgccacccaaactgccaagctccctacgcaaactttttctgagcaacacccagatcaaatacattagtgaagaagatttcaagggattgataaatttaacattactagatttaagcgggaactgtccgaggtgcttcaatgccccatttccatgcgtgccttgtgatggtggtgcttcaattaatatagatcgttttgcttttcaaaacttgacccaacttcgatacctaaacctctctagcacttccctcaggaagattaatgctgcctggtttaaaaatatgcctcatctgaaggtgctggatcttgaattcaactatttagtgggagaaatagcctctggggcatttttaacgatgctgccccgcttagaaatacttgacttgtcttttaactatataaaggggagttatccacagcatattaatatttccagaaacttctctaaacttttgtctctacgggcattgcatttaagaggttatgtgttccaggaactcagagaagatgatttccagcccctgatgcagcttccaaacttatcgactatcaacttgggtattaattttattaagcaaatcgatttcaaacttttccaaaatttctccaatctggaaattatttacttgtcagaaaacagaatatcaccgttggtaaaagatacccggcagagttatgcaaatagttcctcttttcaacgtcatatccggaaacgacgctcaacagattttgagtttgacccacattcgaacttttatcatttcacccgtcctttaataaagccacaatgtgctgcttatggaaaagccttagatttaagcctcaacagtattttcttcattgggccaaaccaatttgaaaatcttcctgacattgcctgtttaaatctgtctgcaaatagcaatgctcaagtgttaagtggaactgaattttcagccattcctcatgtcaaatatttggatttgacaaacaatagactagactttgataatgctagtgctcttactgaattgtccgacttggaagttctagatctcagctataattcacactatttcagaatagcaggcgtaacacatcatctagaatttattcaaaatttcacaaatctaaaagttttaaacttgagccacaacaacatttatactttaacagataagtataacctggaaagcaagtccctggtagaattagttttcagtggcaatcgccttgacattttgtggaatgatgatgacaacaggtatatctccattttcaaaggtctcaagaatctgacacgtctggatttatcccttaataggctgaagcacatcccaaatgaagcattccttaatttgccagcgagtctcactgaactacatataaatgataatatgttaaagttttttaactggacattactccagcagttccctcgtctcgagttgcttgacttacgtggaaacaaactactctttttaactgatagcctatctgactttacatcttcccttcggacactgctgctgagtcataacaggatttcccacctaccctctggctttctttctgaagtcagtagtctgaagcacctcgatttaagttccaatctgctaaaaacaatcaacaaatccgcacttgaaactaagaccaccaccaaattatctatgttggaactacacggaaacccctttgaatgcacctgtgacattggagatttccgaagatggatggatgaacatctgaatgtcaaaattcccagactggtagatgtcatttgtgccagtcctggggatcaaagagggaagagtattgtgagtctggagctgacaacttgtgtttcagatgtcactgcagtgatattatttttcttcacgttctttatcaccaccatggttatgttggctgccctggctcaccatttgttttactgggatgtttggtttatatataatgtgtgtttagctaaggtaaaaggctacaggtctctttccacatcccaaactttctatgatgcttacatttcttatgacaccaaagatgcctctgttactgactgggtgataaatgagctgcgctaccaccttgaagagagccgagacaaaaacgttctcctttgtctagaggagagggattgggacccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtcttgactgaaaatgattcacggtataacaatatgtatgtcgattccattaagcaatactaactgacgttaagtcatgatttcgcgccataataaagatgcaaaggaatgacatttctgtattagttatctattgctatgtaacaaattatcccaaaacttagtggtttaaaacaacacatttgctggcccacagtttt

SEQ ID NO: 16 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 17 - DNA, 3352 bp, Homo sapiens
aggctggtataaaaatcttacttcctctattctctgagccgctgctgcccctgtgggaagggacctcgagtgtgaagcatccttccctgtagctgctgtccagtctgcccgccagaccctctggagaagcccctgccccccagcatgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccgcaataagctggacctctaccacgagcactcattcacggagctaccgcgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccggctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatagccgtgagccggaatcctgcacggtgccacctccacactcacctcacctctgcctgcctggtctgaccctcccctgctcgcctccctcaccccacacctgacacagagca

SEQ ID NO: 18 - protein, 1032 aa, Homo sapiens [sequence omitted]

SEQ ID NO: 19 - DNA, 3002 bp, Homo sapiens
gtggcttggtattcactggcaggtttcagacatttagatctttcttttaatgactaacaccatgcctatctgtggagaagctggcaacatgtcacacctggaaattgtttttcaacattaatactattatttggcagtaatccagattgcttttgccaccaacctgaagacatatagaggcagaaggacaggaataattctatttgtttcctgttttgaaacttccatctgtaaggctatcaaaaggagatgtgagagagggtattgagtctggcctgacaatgcagttcttaaaccaaaggtccattatgcttctcctctctgagaatcctgacttacctcaacaacggagacatggcacagtagccagcttggagacttctcagccaatgctctgagatcaagtcgaagacccaatatacagggttttgagctcatcttcatcattcatatgaggaaataagtggtaaaatccttggaaatacaatgagactcatcagaaacatttacatattttgtagtattgttatgacagcagagggtgatgctccagagctgccagaagaaagggaactgatgaccaactgctccaacatgtctctaagaaaggttcccgcagacttgaccccagccacaacgacactggatttatcctataacctcctttttcaactccagagttcagattttcattctgtctccaaactgagagttttgattctatgccataacagaattcaacagctggatctcaaaacctttgaattcaacaaggagttaagatatttagatttgtctaataacagactgaagagtgtaacttggtatttactggcaggtctcaggtatttagatctttcttttaatgactttgacaccatgcctatctgtgaggaagctggcaacatgtcacacctggaaatcctaggtttgagtggggcaaaaatacaaaaatcagatttccagaaaattgctcatctgcatctaaatactgtcttcttaggattcagaactcttcctcattatgaagaaggtagcctgcccatcttaaacacaacaaaactgcacattgttttaccaatggacacaaatttctgggttcttttgcgtgatggaatcaagacttcaaaaatattagaaatgacaaatatagatggcaaaagccaatttgtaagttatgaaatgcaacgaaatcttagtttagaaaatgctaagacatcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacacatgcttttcccgaattatcctacgaaattccaatatttaaattttgccaataatatcttaacagacgagttgtttaaaagaactatccaactgcctcacttgaaaactctcattttgaatggcaataaactggagacactttctttagtaagttgctttgctaacaacacacccttggaacacttggatctgagtcaaaatctattacaacataaaaatgatgaaaattgctcatggccagaaactgtggtcaatatgaatctgtcatacaataaattgtctgattctgtcttcaggtgcttgcccaaaagtattcaaatacttgacctaaataataaccaaatccaaactgtacctaaagagactattcatctgatggccttacgagaactaaatattgcatttaattttctaactgatctccctggatgcagtcatttcagtagactttcagttctgaacattgaaatgaacttcattctcagcccatctctggattttgttcagagctgccaggaagttaaaactctaaatgcgggaagaaatccattccggtgtacctgtgaattaaaaaatttcattcagcttgaaacatattcagaggtcatgatggttggatggtcagattcatacacctgtgaataccctttaaacctaaggggaattaggttaaaagacgttcatctccacgaattatcttgcaacacagctctgttgattgtcaccattgtggttattatgctagttctggggttggctgtggccttctgctgtctccactttgatctgccctggtatctcaggatgctaggtcaatgcacacaaacatggcacagggttaggaaaacaacccaagaacaactcaagagaaatgtccgattccacgcatttatttcatacagtgaacatgattctctgtgggtgaagaatgaattgatccccaatctagagaaggaagatggttctatcttgatttgcctttatgaaagctactttgaccctggcaaaagcattagtgaaaatattgtaagcttcattgagaaaagctataagtccatctttgttttgtctcccaactttgtccagaatgagtggtgccattatgaattttactttgcccaccacaatctcttccatgaaaattctgatcatataattcttatcttactggaacccattccattctattgcattcccaccaggtatcataaactgaaagctctcctggaaaaaaaagcatacttggaatggcccaaggataggcgtaaatgtgggcttttctgggcaaaccttcgagctgctattaatgttaatgtattagccaccagagaaatgtatgaactgcagacattcacagagttaaatgaagagtctcgaggttctacaatctctctgatgagaacagattgtctataaaatcccacagtccttgggaagttggggaccacatacactgttgggatgtacattgatacaacctttatgatggcaatttg

SEQ ID NO: 20 - protein, 811 aa, Homo sapiens
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGIRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

SEQ ID NO: 21 - DNA, 215 bp, Homo sapiens
aaaaacaaaacatttgagaaacacggctctaaactcatgtaaagagtgcatgaaggaaagcaaaaacagaaatggaaagtggcccagaagcattaagaaagtggaaatcagtatgttccctatttaaggcatttgcaggaagcaaggccttcagagaacctagagcccaaggttcagagtcacccatctcagcaagcccagaagtatctgcaata

SEQ ID NO: 22 - DNA, 36 bp, Artificial
  5′ primer for human IFN-alpha promoter
acgagatctaagcttaaaacaaaacatttgagaaac

SEQ ID NO: 23 - DNA, 28 bp, Artificial
  3′ primer for human IFN-alpha promoter
acgagatctagatattgcagatacttct